(19) United States
(12) Patent Application Publication
(10) Pub. No.: US 2012/0078527 A1
(43) Pub. Date: Mar. 29, 2012

(54) KIT AND METHOD FOR PREDICTING SENSITIVITY OF GASTRIC CANCER PATIENT TO ANTI-CANCER AGENT

(75) Inventors: Chang-eun YOO, Seoul (KR); Tae-jin AHN, Seoul (KR); Kyu-sang LEE, Ulsan (KR); Jeo-young SHIM, Yongin-si (KR); Ki-eun KIM, Seoul (KR); Myoung-soon KIM, Seoul (KR); Sung-woo HONG, Seoul (KR); Tae-han JEON, Hwaseong-si (KR); Jhin-gook KIM, Seoul (KR); Se-hoon PARK, Seoul (KR); In-suk SOHN, Seoul (KR); Sin-ho JUNG, Chapel Hill, NC (US)

(73) Assignee: SAMSUNG ELECTRONICS CO., LTD., Suwon-si (KR)

(21) Appl. No.: 13/244,095

(22) Filed: Sep. 23, 2011

(30) Foreign Application Priority Data
Sep. 27, 2010 (KR) 10-2010-0093301

(51) Int. Cl. G06F 19/00; C12Q 1/68; C40B 30/04

(52) U.S. Cl. 702/19; 506/9; 435/6.11

(57) ABSTRACT

A kit and method for predicting the sensitivity of gastric cancer patient to an anti-cancer agent are disclosed.

### CROSS-REFERENCE TO RELATED APPLICATIONS

This application claims priority to Korean Patent Application No. 10-2010-0093301, filed on Sep. 27, 2010, and all the benefits accruing therefrom under 35 U.S.C. §119, the disclosure of which is incorporated by reference.

### BACKGROUND

1. Field

The present disclosure relates to kits and methods for predicting the sensitivity of a gastric cancer patient to an anti-cancer agent.

2. Description of the Related Art

Gastric cancer is one of the most incident cancers in Korea, along with lung and liver cancers, with a mortality of about 20.9 per one hundred thousand people. At its early stages, gastric cancer is known to be indistinguishable from common gastric diseases since no or only nonspecific gastrointestinal, symptoms appear. As gastric cancer progresses, patients may experience weight loss, abdominal pain, vomiting, bleeding, and the like. The best treatment for gastric cancer known so far is to surgically excise tumor sites, and in this regard, various surgical excisions are available. For a complete recovery, a tumor site as large as possible may be surgically removed with consideration of the range of the surgical resection that may not cause aftereffects following the surgery. However, if there is a considerable advance or metastasis to other organs, radical curative surgery may not be applicable or may be too risky as compared to predicted operation effects. Then, other treatment methods, for example, medication with anti-cancer agents, may be applicable.

Anti-cancer agents may be effective in gastric cancer treatment. However, side effects, such as reduced blood cells, hypersensitivity, nausea, vomiting, or alopecia, occur when they are administered to patients, which hinders predicting the therapeutic effects of the anti-cancer agents. Research into anticancer agent sensitivity has been conducted so far to detect a tumor maker that is delivered through the blood, and thus, the reactivity of the tumor itself to the anti-cancer agent cannot yet be accurately determined.

Therefore, there is a need for a method to predict accurately the sensitivity of a gastric cancer patient to an anti-cancer agent, which may help the patient not to suffer from unnecessary side effects caused by the anti-cancer agent and may also reduce unnecessary medical expenses.

### SUMMARY

Provided are kits for predicting the sensitivity of a gastric cancer patient to an anti-cancer agent, and methods for using the kits.

### BRIEF DESCRIPTION OF THE DRAWINGS

These and/or other aspects will become apparent and more readily appreciated from the following description of the embodiments, taken in conjunction with the accompanying drawings of which:

FIG. 1 is a graph plotting the principal component values (PC1, PC2) determined for a gastric cancer patient population by a principal component analysis obtained using genotype data of 300 single nucleotide polymorphisms (SNP), wherein 0 indicates data for patients sensitive to the anti-cancer agent, and X indicates data for patients insensitive to the anti-cancer agent; and

FIG. 2 is a graph illustrating leave-one-out cross-validation results for percent accuracy of the predictions of patient sensitivity to the anti-cancer agent made using a linear discriminant analysis (LDA) model as a function of the number of SNPs included in the LDA model.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present invention.

According to aspects of the present invention, there are provided a kit and a method for predicting the sensitivity of a gastric cancer patient to an anti-cancer agent.

According to an embodiment of the present invention, there is provided a kit for predicting the sensitivity of a gastric cancer patient to an anti-cancer agent. The kit includes polynucleotides having nucleotide sequences of SEQ ID NOS: 1 to 59, or complements thereof, each of which includes a single nucleotide polymorphism (SNP) at position 27.

The term “single nucleotide polymorphism (SNP)” used herein refers to a single-nucleotide variation between individuals of the same species and is used as known in the art. It is estimated that human SNPs occur at a frequency of 1 in every 1,000 bp.

The term “nucleotide” used herein is a molecule made up of a nitrogenous base, a sugar, and at least one phosphate group, and includes natural nucleotides or nucleotide analogues in which a sugar, base, or phosphate is modified unless otherwise stated (Scheit, Nucleotide Analogs, John Wiley, New York 1980; Uhlman and Peyman, Chemical Reviews, 90:543-584 1990). The term “polynucleotide” used herein refers to a polymer of the nucleotides. Polynucleotides include polydeoxyribonucleotides and polyribonucleotides, as well as polymers of nucleotides including nucleotide analogues. Polynucleotides can be in single- or double-stranded forms. For example, a polynucleotide can be a double- or single-stranded polydeoxyribonucleotide, a double- or single-stranded polyribonucleotide, or a hybrid duplex of a single-stranded polydeoxyribonucleotide and a single-stranded polyribonucleotide.

The polynucleotide may include 10 to 100 or 10 to 50 nucleotides containing a SNP site and having a nucleotide sequence selected from the group consisting of nucleotide sequences of SEQ ID NOS: 1 to 58, or complements thereof. In this regard, the SNP site of each of the nucleotide sequences of SEQ ID NOS: 1 to 59 is at position 27.

The polynucleotides having nucleotide sequences of SEQ ID NOS: 1 to 59, each with a polymorphic site at position 27, are reference sequences for identification of the various genomic polymorphic sites (see Table 3) shown herein to be associated with the sensitivity of gastric cancer patients to an anti-cancer agent. This association may be identified by administering the anti-cancer agent to gastric cancer patients, and comparing the nucleotide sequence of genomic DNA obtained from biological samples of patients who are determined to be sensitive or not sensitive to the anti-cancer agent, based on showing decreased tumor size subsequent to treatment with the anti-cancer agent. The sequence comparison may be performed by immobilizing polynucleotides to detect each of the alleles of a given SNP on a microarray chip, and hybridizing DNA obtained from the biological samples of the patients, who are sensitive or not sensitive to the anti-cancer agent, to the DNA immobilized on the microarray to genotype the patients at the SNP.

Furthermore, if an allelic nucleotide of a SNP is found in double-stranded genomic DNA, it may be construed that the SNP includes a nucleotide complementary to the nucleotide in the complementary strand of the DNA. For example, in the complementary strand, the nucleotide “T” of the SNP would be the complementary “A”.

According to an embodiment, the sensitivity of a gastric cancer patient to an anti-cancer agent may be predicted using a kit including the polynucleotides having nucleotide sequences of SEQ ID NOS: 1 to 59, or complements thereof. For example, whether to administer the anti-cancer agent to the gastric cancer patient may be easily determined by extracting DNA from the gastric cancer patient before administering the anti-cancer agent to the gastric cancer patient, contacting the patient's DNA with the polynucleotides having nucleotide sequences of SEQ ID NOS: 1 to 59, or a complement thereof, under conditions permitting hybridization, and analyzing the hybridization results. Analyzing the hybridization results can result in determination of the patient's genotype at the SNPs tested with the polynucleotides, which can be further used to predict the patient's sensitivity to the anti-cancer agent. The analysis of the results will be described later. Examples of the anti-cancer agent include cisplatin.

According to an embodiment, the polynucleotides may be immobilized on a microarray.

The term “microarray” used herein refers to a substrate on which a group of polynucleotides is densely immobilized in a predetermined region. Such a microarray is well known in the art. For example, microarrays are disclosed in U.S. Pat. Nos. 5,445,934 and 5,744,305, the contents of which are entirely incorporated herein by reference.

The polynucleotides having nucleotide sequences of SEQ ID NOS: 1 to 59, or a complement thereof, may be used as hybridizable array elements and may be immobilized onto a substrate. The substrate may be a solid or semi-solid support including, for example, a membranes filter, chip, slide, wafer, fiber, magnetic bead or nonmagnetic bead, gel, tube, plate, polymer, microparticle, and capillary. The immobilization may be achieved by noncovalent binding or covalent binding, for example, using UV rays. For example, the polynucleotides may be bound to the surface of glass that is modified to contain an epoxy compound or an aldehyde group, or to a polylysine-coated substrate surface using UV rays. In addition, the polynucleotides may be bound to the substrate by a linker, such as, an ethylene glycol oligomer or a diamine.

According to another embodiment of the present invention, there is provided a method of predicting the sensitivity of a gastric cancer patient to an anti-cancer agent. The method includes: obtaining a biological sample from the gastric cancer patient; identifying the genotype of a SNP contained in the biological sample with the polynucleotides of the kit; and determining the sensitivity of the gastric cancer patient to the anti-cancer agent based on the genotype data using statistical classification analysis.

According to an embodiment, the statistical classification analysis may be selected from the group consisting of linear discriminant analysis, principal component analysis, quantitative descriptive analysis, logistic regression analysis, support vector machine analysis, and LASSO analysis. These statistical classification analyses are well known in the art, and thus descriptions thereof will not be provided herein.

According to an embodiment, the statistical classification analysis may include determining principal component analysis values PC1 and PC2 based on the identified SNP genotype data for a patient using Equations I and II; and determining the sensitivity of the gastric cancer patient to the anti-cancer agent by applying the PC1 and PC2 values to a linear discriminant analysis model of the SNPs that can be genotyped by the polynucleotides contained in the kit.

$$\begin{matrix}PC 1 = {\sum }_{i = 1}^{\# of SNPs} {c}_{1 i} \cdot {SNP}_{i} & Equation I \\ PC 2 = {\sum }_{i = 1}^{\# of SNPs} {c}_{2 i} \cdot {SNP}_{i} & Equation II\end{matrix}$$

In Equations I and II, SNPi is a genotype of the i^{th} biallelic SNP, c₁ᵢ is a contribution degree (coefficient) of the i^{th} SNP in the first component obtained in the principal component analysis, and c₂ᵢ is a contribution degree (coefficient) of the i^{th} SNP in the second component obtained in the principal component analysis. In the PCA, the patient genotype at each biallelic SNP is encoded as 0, 1, or 2, depending on the number of minor alleles present in the genotype. For each SNP, the minor (B) allele is the allele in the NCBI dbSNP database designated as the minor allele. PCA was performed using the computer program, R software 2.11 version (Source: R Development Core Team, Regnow).

The method of predicting the sensitivity of a gastric cancer patient to an anti-cancer agent will now be described in detail.

The method includes obtaining a biological sample from the gastric cancer patient.

The biological sample may be any sample including cells obtained from the gastric cancer patient. For example, the biological sample may include blood, lymph, plasma, serum, urine, tissue, cell, organ, bone marrow, saliva, sputum, cerebrospinal fluid, or the like, but is not limited thereto. The biological sample may be a gastric tumor tissue removed by surgical resection, or any of various methods, for example, optical resection using a laser. The biological sample may be obtained from the gastric cancer patient when the type of anti-cancer therapeutic method for the patient is determined, i.e., when it is determined to administer an anti-cancer agent.

The method may include identifying the genotype of a SNP present in the sample with a polynucleotide of the kit.

As described above, the kit includes polynucleotides having nucleotide sequences of SEQ ID NOS: 1 to 59, or complements thereof. The polynucleotides are reference sequences for SNPs associated with the sensitivity to the anti-cancer agent. The genotype of the SNP in a patient may be identified by extracting DNA from the gastric cancer patient to receive the anti-cancer agent, and hybridizing the DNA to the polynucleotides of the kit.

The hybridization may be performed by controlling hybridization conditions, such as temperature, concentrations of components of the buffer solution, hybridizing and washing times, pH and ionic strength of the buffer solution, and the hybridization conditions may vary according to various factors such as the length and GC content of a probe polynucleotide, and a target nucleotide sequence. Hybridization conditions are disclosed by Joseph Sambrook, et al., Molecular Cloning, A Laboratory Manual, Cold Spring Harbor Laboratory Press, Cold Spring Harbor, N.Y. 2001; and M. L. M. Anderson, Nucleic Acid Hybridization, Springer-Verlag New York Inc. N.Y. 1999. For example, among stringent conditions disclosed in the above documents, high stringency conditions include hybridizing at 65° C. using 0.5 M NaHPO₄, 7% sodium dodecyl sulfate (SDS), and 1 mM EDTA, and washing with 0.1× standard sodium citrate (SSC)/0.1°)/0 SDS at 68° C. For example, low stringency conditions include washing with 0.2×SSC/0.1°)/0 SDS at 42° C.

A signal may be detected to identify whether hybridization occurs. The signal may be detected using various methods according to the detectable label bound to the polynucleotide serving as a probe. The “detectable label” used herein refers to an atom or molecule used to specifically detect a molecule including the label, from among the same type of molecules without the label. For example, the detectable label may include a colored bead, an antigen determinant, enzyme, hybridizable nucleic acid, a chromophore, a fluorescent material, a phosphorescent material, an electrically detectable molecule, a molecule providing modified fluorescence-polarization or modified light-diffusion, or a quantum dot. In addition, the detectable label may be radioactive isotopes such as P³² and S³⁵, a chemiluminescent compound, labeled binding protein, a heavy metal atom, a spectroscopic marker such as a dye, or a magnetic label. The dye may be a quinoline dye, a triarylmethane dye, phthalene, an azo dye, or a cyanine dye, but is not limited thereto. The fluorescent material may be Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, mcheery, Oregon Green, Oregon Green 488-X, Oregon Green, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3 or thiazole orange, but is not limited thereto. The genotype of a SNP associated with a patient's sensitivity to the anti-cancer agent may be identified by analyzing the hybridization signals generated by the hybridization. In other words, SNP genotype data may be obtained by analyzing signals generated by hybridizing the DNA contained in the biological sample to the polynucleotides of the kit. The SNP genotype data may be used in the following stages.

Then, the method may include determining principal component analysis values PC1 and PC2 for the patient using the identified SNP genotype data, as shown in Equations I and II.

$$\begin{matrix}PC 1 = {\sum }_{i = 1}^{\# of SNPs} {c}_{1 i} \cdot {SNP}_{i} & Equation I \\ PC 2 = {\sum }_{i = 1}^{\# of SNPs} {c}_{2 i} \cdot {SNP}_{i} & Equation II\end{matrix}$$

In Equations I and II, SNPi is a genotype of the i^{th} SNP, c₁ᵢ is a contribution degree (coefficient) of the i^{th} SNP in the first component obtained from the principal component analysis, and c₂ᵢ is a contribution degree (coefficient) of the i^{th} SNP in the second component obtained from the principal component analysis.

Finally, the method may include determining whether the gastric cancer patient is sensitive to the anti-cancer agent by applying the determined PC1 and PC2 values to a linear discriminant analysis model with respect to the SNPs genotyped by the polynucleotides contained in the kit.

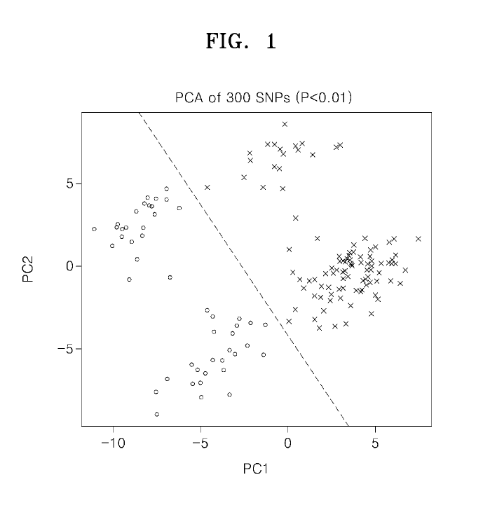

For example, whether a gastric cancer patient is sensitive to the anti-cancer agent may be determined based on the positions of the PC1 and PC2 of the patient in an x-y plane. For example, for the data of Example 1 illustrated in FIG. 1, patients who are nonresponsive to the anti-cancer drug and patients who are responsive to the anti-cancer drug are found in different areas of the PC1-PC2 graph, separated by the dotted line. Thus determination of the PC1 and PC2 values of a patient permit prediction of the patient's sensitivity to the anti-cancer drug based on the location of the patient's PC1-PC2. values on the graph. Linear discriminant analysis is a widely known technique used to obtain a linear discriminant that may divide data on a plane into two groups, and thus the descriptions thereof will be omitted herein.

The present invention will be described in further detail with reference to the following examples. These examples are for illustrative purposes only and are not intended to limit the scope of the invention.

### Example 1

### Determination of SNPs Associated with Sensitivity of Gastric Cancer Patients to an Anti-Cancer Agent

A population of 151 advanced (terminal) gastric cancer patients who were treated with cisplatin-based chemotherapy (in Samsung Medical Center, Seoul, Korea) after palliative surgical resection were selected to test for sensitivity to the anti-cancer agent. That is, the anti-cancer agent (cisplatin) was administered to the patients according to National Comprehensive Cancer Network (NCCN) guidelines, and changes in tumor size after the administration of the anti-cancer agent were measured to determine whether the anti-cancer therapy was effective. Then, the patients were grouped into two groups: one group of 103 patients for which the anti-cancer therapy was effective (responders), and the other group of 48 patients for which the anti-cancer therapy was not effective (nonresponders). In addition, to identify SNPs associated with response or nonresponse to the anti-cancer agent, DNA of each of the patients was extracted from gastric tumor tissue removed from the patient using QIAamp DNA Mini and blood Mini kits.

Microarray chips for identifying SNPs associated with sensitivity to anti-cancer agents were manufactured according to the following process. First, SNPs registered in the National Cancer Institute (NCI) Cancer SNP database and the Pharm GKB database (T. E. Klein, et al., “Integrating Genotype and Phenotype Information: An Overview of the PharmGKB Project” (220 k PDF), The Pharmacogenomics Journal (2001) 1, 167-170) were selected for testing. Polynucleotide sequences (probes) to detect each of the alleles of the selected SNPs were immobilized on 15 wafers using a general photolithography method to manufacture the microarray chips. In the microarray chips, ProcessQC AD was 1.76, and CV was 3.15% on average.

The probes immobilized onto the microarray chips were hybridized with the extracted DNA samples of all the patients to genotype the patient at each of the SNPs in order to identify which of the 93,137 SNPs were associated with sensitivity to the anti-cancer agent. Then, a Max Test method was applied to the patient genotype data for the SNPs. The Max Test method will be described as follows.

In the MAX Test method for each SNP, a plurality of genetic models was tested for the significance of the association of SNP genotypes of the subjects with anti-cancer agent response or nonresponse to determine the genetic model classification of the SNP by determining the maximum significance among the tested models. The genetic models, which are for statistically testing the genetic characteristics of the SNPs, may include a dominant model, a recessive model, and an additive model. In this regard, the significances determined include the significance of whether the subjects can be significantly classified into either a responder group or a nonresponder group in a specific condition according to the SNPs and the significance of each genetic model used to test genetic characteristics of the SNPs. The most significant SNPS, determined for any of the 3 genetic models, were selected for prediction modeling. Although tens of thousands or hundreds of thousands of SNPs in the subjects may have allelic variation, some of the variation at SNPs may not be associated with sensitivity to the anti-cancer agent. That is, some of the SNPs of the patients may not be associated or may be insignificantly associated with sensitivity to the anti-cancer agent. Thus, such SNPs may not be considered in the statistical models for predicting response or nonresponse to the anti-cancer agent. Accordingly, statistically analyzing genotype data of the SNPs as shown in Table 1 below permits determination of SNPs at which genotypic variation is significantly associated with anti-cancer drug sensitivity and which genotypes show that significant association.

TABLE 1

| SNP 1 | AA | AB | BB | Total |
|---|---|---|---|---|
| Response | x0 | x1 | x2 | x |
| No Response | n0 − x0 | n1 − x1 | n2 − x2 | n − x |
| Total | n0 | n1 | n2 | n |

In Table 1, AA, AB and BB represent the three possible genotypes that can occur for biallelic SNP1 having A and B as the two possible alleles at the site. Response and No Response respectively indicate patient response or no response to the anti-cancer agent. In more detail, the classification into Response and No Response indicates a classification of the patients treated with the anti-cancer agent into a responder group and a nonresponder group, and subsequently also classifying all patients according to their determined genotypes at SNP1. Each of the x0 to x2 indicates the number of each of the AA, AB and BB genotypes in the genotype data of the subjects who are in the responder group (Response). In addition, each of n0 to n2 indicates the total number of each of the AA, AB and BB genotypes determined in the overall patient group. Accordingly, the number of each of the AA, AB and BB genotypes in the genotype data of the subjects who are in the nonresponder group (No Response) is n0-x0, n1-x1 and n2-x2, respectively.

By using the MAX Test method, a group of SNPs with a genotype significantly associated with response to the anti-cancer agent and a group of SNPs with a genotype significantly associated with nonresponse to the anti-cancer agent were selected according to p-values as shown in Table 2 below.

TABLE 2

| p-values | <0.01 | <0.001 | <0.0005 |
|---|---|---|---|
| Number of SNP | 300 | 26 | 11 |

### Example 2

### Statistical Model for Predicting Sensitivity of Gastric Cancer Patient to the Anti-Cancer Agent

A statistical model for predicting sensitivity of gastric cancer patients to the anti-cancer agent was obtained by performing principal component analysis (PCA) on the patient population of Example 1 using the 300 SNPs (p≦0.01) selected from among the SNPs tested in Example 1 (see FIG. 1). In FIG. 1, PC1 and PC2 are the principal component analysis values for each of the patients, obtained using Equations I and II below with respect to the 300 SNPs shown to have a genotype significantly associated with response or nonresponse to the anti-cancer agent.

$$\begin{matrix}PC 1 = {\sum }_{i = 1}^{\# of SNPs} {c}_{1 i} \cdot {SNP}_{i} & Equation I \\ PC 2 = {\sum }_{i = 1}^{\# of SNPs} {c}_{2 i} \cdot {SNP}_{i} & Equation II\end{matrix}$$

In Equations I and II, SNPi is a genotype of the i^{th} SNP, C₁ᵢ is a contribution degree (coefficient) of the i^{th} SNP in the first component as a result of the principal component analysis, and c₂ᵢ is a contribution degree (coefficient) of the i^{th} SNP in the second component as a result of the principal component analysis.

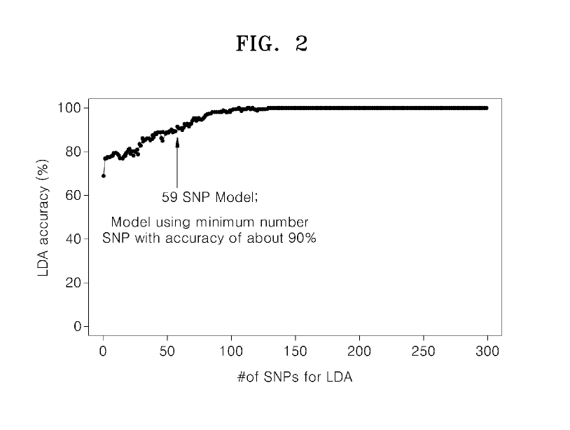

In addition, the accuracy of prediction of response or nonresponse to the anti-cancer agent using genotype data for the 300 SNPs was 100% when a leave-one-out cross-validation was performed using linear discriminant analysis (see FIG. 2). Based on the results, 300 SNPs were sequentially removed from the SNP having the lowest coefficient and cross-validation was performed using the linear discriminant analysis in order to obtain a predictive model for sensitivity of the gastric cancer patients to the anti-cancer agent using the minimum number of SNPs. The accuracy data are shown in FIG. 2. As a result, a statistical model using a minimum number of SNPs, 59, with about 90% accuracy was obtained. NCBI dbSNP Accession Nos. and principal component analysis values of the 59 SNPs in the minimal model are listed in Table 3 below. Reference polynucleotide sequences for each of the 59 SNPs shown in Table 3 are sequentially listed in SEQ ID NOS: 1 to 59.

TABLE 3

| | | | Genetic | A | B |
|---|---|---|---|---|---|
| id | c₁ᵢ | c₂ᵢ | model | allele | allele |
| rs389221 | −10.05027513 | 1.453727459 | Recessive | C | T |
| rs450818 | −10.29141379 | 1.700777026 | Recessive | A | T |
| rs9371537 | −3.674975345 | −4.227444406 | Recessive | C | T |
| rs10759122 | 7.432672997 | −0.369177276 | Additive | C | G |
| rs436563 | −10.49095645 | 2.2733324 | Recessive | A | T |
| rs2779407 | 2.46001556 | 5.408686166 | Additive | C | T |
| rs6904133 | 3.58589819 | 2.921444398 | Dominant | G | T |
| rs1522986 | 3.76092368 | 1.995092585 | Dominant | A | T |
| rs452582 | 10.92609749 | −2.695203868 | Dominant | C | G |
| rs1856859 | 4.429475155 | 3.232893592 | Dominant | A | C |
| rs4263255 | 3.222935509 | 3.156373306 | Dominant | C | T |
| rs440834 | −10.1302067 | 1.431159686 | Recessive | A | C |
| rs832262 | −10.42654454 | 2.148725107 | Recessive | C | T |
| rs4468852 | −2.255327715 | −3.312021085 | Additive | A | C |
| rs2779400 | 1.687492924 | 6.100066913 | Additive | A | T |
| rs9371538 | 4.101462357 | 3.555535893 | Dominant | A | T |
| rs2417957 | −2.538619622 | −7.426488134 | Additive | C | T |
| rs12141182 | −0.846768359 | −5.857861579 | Additive | C | T |
| rs17526478 | 2.757576041 | 3.102375765 | Additive | A | C |
| rs381343 | 10.82882988 | −2.256677602 | Dominant | C | G |
| rs2219828 | −1.953307253 | −6.524060838 | Recessive | C | T |
| rs10754593 | −0.920831444 | −6.054461222 | Dominant | C | G |
| rs367461 | 10.20863574 | −3.123206755 | Dominant | A | T |
| rs6912538 | −1.634954567 | −5.670218868 | Additive | A | C |
| rs159227 | 10.76341429 | −2.960812358 | Dominant | C | G |
| rs1726597 | −0.474523006 | −4.885480566 | Dominant | A | G |
| rs11045808 | 3.246417694 | 7.433920157 | Additive | C | G |
| rs2805410 | 1.590483952 | 5.799199642 | Additive | A | G |
| rs3120683 | −0.690286449 | −5.547864506 | Dominant | A | C |
| rs150409 | −8.556107872 | 1.507419838 | Recessive | C | T |
| rs10119035 | 7.49680662 | −1.51850998 | Dominant | G | T |
| rs1945427 | −2.946128342 | −2.791791044 | Recessive | A | G |
| rs11045834 | −1.892213212 | −8.320852154 | Additive | C | T |
| rs4149070 | −1.399366099 | −6.763288871 | Recessive | C | G |
| rs7513677 | −0.097482608 | −6.39504963 | Additive | C | T |
| rs7612183 | 2.127192474 | 3.53936427 | Recessive | C | T |
| rs439467 | 10.46552012 | −2.648558968 | Dominant | C | T |
| rs10925334 | −0.779592751 | −5.858711684 | Dominant | A | T |
| rs404317 | −9.983162268 | 2.186853743 | Recessive | A | G |
| rs11849883 | 3.890264043 | 2.167343659 | Recessive | A | G |
| rs3213829 | 1.594284803 | 3.655927479 | Recessive | G | T |
| rs424301 | −8.033828082 | 0.904835818 | Recessive | A | G |
| rs4869742 | 2.687271548 | 4.498410595 | Recessive | C | T |
| rs4149045 | −0.996443482 | −8.103224573 | Additive | A | G |
| rs10739208 | −5.398623786 | 1.307836787 | Recessive | C | T |
| rs16863204 | 4.209399265 | 1.54566897 | Dominant | C | T |
| rs450485 | −10.21333403 | 2.609230751 | Recessive | A | G |
| rs291296 | −8.453608898 | 1.311237749 | Recessive | A | G |
| rs12427008 | 2.59302837 | 5.848214189 | Dominant | C | T |
| rs2490366 | −0.742346528 | −5.891732591 | Dominant | A | T |
| rs11753987 | 2.528306816 | 4.108136234 | Additive | C | T |
| rs7588062 | 2.411033045 | 2.702351304 | Additive | C | T |
| rs4149068 | 2.200508605 | 6.024315255 | Dominant | C | T |
| rs7039475 | −6.791847666 | 1.187127131 | Recessive | A | T |
| rs1361116 | 1.818665376 | 5.183887581 | Additive | G | T |
| rs10733202 | −6.197962343 | 1.71922093 | Additive | G | T |
| rs4149031 | 3.275450991 | 5.772561686 | Dominant | C | G |
| rs1945428 | 3.410399186 | 2.219218808 | Dominant | A | C |
| rs7034072 | 7.150575603 | −2.509774316 | Additive | G | T |

Table 4 below shows whether sensitivity of a gastric cancer patient to the anti-cancer agent is predictable using the 59 SNP statistical model. The accuracy of prediction with the optimized model using the 59 SNPs may be represented by a percentage of the number of predicted patient responses that are identical to the number of observed patient responses with respect to the total patient sample. The accuracy of prediction of anti-cancer agent sensitivity is represented by [(151-13)/151×100], which is equal to 91.39%.

TABLE 4

| | | Predicted response | | |
|---|---|---|---|---|
| | | Anti-cancer | Anti-cancer | |
| Data | | agent(−) | agent (+) | Total Observed |
| Observed | Anti-cancer | 98 | 5 | 103 |
| response | agent (−) | | | |
| | Anti-cancer | 8 | 40 | 48 |
| | agent (+) | | | |

| | |
|---|---|
| Overall accuracy | 91.39% |

The statistical models used in Examples 1 and 2 to obtain the predictive model for the method are commonly used in statistical fields and will be known to one of ordinary skill in the art.

As described above, according to one or more of the above embodiments of the present invention, the sensitivity of a gastric cancer patient to an anti-cancer agent may be efficiently predicted using a biological sample of the gastric cancer patient by using the kit and method for predicting a gastric cancer patient's sensitivity to the anti-cancer agent.

The terminology used herein is for the purpose of describing particular embodiments only and is not intended to be limiting of the invention. The terms “a” and “an” do not denote a limitation of quantity, but rather denote the presence of at least one of the referenced item. The terms “comprising”, “having”, “including”, and “containing” are to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A kit for predicting the sensitivity of a gastric cancer patient to an anti-cancer agent, the kit comprising
polynucleotides having nucleotide sequences of SEQ ID NOS: 1 to 59, or the complement thereof, each of which includes a single nucleotide polymorphism (SNP) at position 27.

2. The kit of claim 1, wherein the polynucleotides are immobilized on a microarray.

3. A method of predicting the sensitivity of a gastric cancer patient to an anti-cancer agent, the method comprising:
obtaining a biological sample from the gastric cancer patient;
identifying in the biological sample the patient's genotype at a SNP contained in the kit of claim 1; and
determining the sensitivity of the gastric cancer patient to the anti-cancer agent by using statistical classification analysis of the identified SNP genotype.

4. The method of claim 3, wherein the statistical classification analysis is selected from the group consisting of linear discriminant analysis, principal component analysis, quantitative descriptive analysis, logistic regression analysis, support vector machine analysis, and LASSO analysis.

5. The method of claim 3, wherein the statistical classification analysis comprises:
determining principal component analysis values PC1 and PC2 based on the identified SNP genotype data using Equations I and II and the coefficients of Table 3; and
determining the sensitivity of the gastric cancer patient to the anti-cancer agent by applying the PC1 and PC2 values to a linear discriminant analysis model with respect to the SNP,
$$\begin{matrix}PC 1 = {\sum }_{i = 1}^{\# of SNPs} {c}_{1 i} \cdot {SNP}_{i} & Equation I \\ PC 2 = {\sum }_{i = 1}^{\# of SNPs} {c}_{2 i} \cdot {SNP}_{i} & Equation II\end{matrix}$$
wherein SNPi is a genotype of the i^{th} SNP, c₁ᵢ is a contribution degree of the i^{th} SNP in a first component obtained from the principal component analysis, and c₂ᵢ is a contribution degree of the i^{th} SNP in a second component obtained from the principal component analysis.

6. The method of claim 3, wherein the biological sample comprises a gastric tumor tissue.

7. The method of claim 3, wherein the anti-cancer agent is cisplatin.


SEQUENCE LISTING (59 sequences)

SEQ ID NO: 1 - DNA, 52 bp, Homo sapiens
aatattaatctgaaacagttaaacttyaaagtcattatgtgttacatgataa

SEQ ID NO: 2 - DNA, 52 bp, Homo sapiens
cattagtaatcgtggactacttcaaawgcaagaatattagcattactgattt

SEQ ID NO: 3 - DNA, 52 bp, Homo sapiens
ccactgctgcatctgaaagttaactayaatgccggataccatctgtggtggt

SEQ ID NO: 4 - DNA, 52 bp, Homo sapiens
cctattactcagttactgttaatttasattccacttgaacattatcagtgac

SEQ ID NO: 5 - DNA, 52 bp, Homo sapiens
gaaaattgaagcttaaatctattaggwaacttgtcaatgaatcaaataataa

SEQ ID NO: 6 - DNA, 52 bp, Homo sapiens
ttattttaaaatgtatgattgaattaytactgaccatagtcaccctgttgtg

SEQ ID NO: 7 - DNA, 52 bp, Homo sapiens
aatctgtgtgcatatatatttaggagkatagctaaatcttgttgaattgagc

SEQ ID NO: 8 - DNA, 52 bp, Homo sapiens
cacaacaggctaggcccagatgaggawgatagaattacagtgaaggaggagg

SEQ ID NO: 9 - DNA, 52 bp, Homo sapiens
gacattttatgaaagatagtttagaasaaaaaatggtatcacaactaaacta

SEQ ID NO: 10 - DNA, 52 bp, Homo sapiens
gtgtgagccgccatgcccggccatacmtacaatgtttaaggagtttttgctg

SEQ ID NO: 11 - DNA, 52 bp, Homo sapiens
gaataaatgcagctacagaattctttyactctagaagataaaaagaataagc

SEQ ID NO: 12 - DNA, 52 bp, Homo sapiens
atagtgtcatcctaggatgtagtatcmggtaagtagagtatggctagtttct

SEQ ID NO: 13 - DNA, 52 bp, Homo sapiens
taaacaaaaatcaggtacagaaagctycagattattgctagaaggatgtcaa

SEQ ID NO: 14 - DNA, 52 bp, Homo sapiens
catgtcaggactttaaggccctagttmagttctcaggattgtatttgcagtg

SEQ ID NO: 15 - DNA, 52 bp, Homo sapiens
ggctgaaaactagcatttaaccagtawctagtaactctgtgcgagctattgg

SEQ ID NO: 16 - DNA, 52 bp, Homo sapiens
accaccaccagcaaagtcgtgctgagwttcctgacccatagaaactgtgaga

SEQ ID NO: 17 - DNA, 52 bp, Homo sapiens
tcaacttaatacaactagttttgataytcttctacattttatgctgtttctt

SEQ ID NO: 18 - DNA, 52 bp, Homo sapiens
aatgtaattacagagcacttataaaayctttcagtactttagcaaccacaac

SEQ ID NO: 19 - DNA, 52 bp, Homo sapiens
tattagtactggaatatttgactgagmaagaactctggtttcagaaagactg

SEQ ID NO: 20 - DNA, 52 bp, Homo sapiens
atctgaactctgttcatatgattaaasatcactgcacttcaccatttcagtt

SEQ ID NO: 21 - DNA, 52 bp, Homo sapiens
tcagttctttattggatatatcatttyaatgtcttctggtctctgatgagaa

SEQ ID NO: 22 - DNA, 52 bp, Homo sapiens
agttattatgtactctttatggactasagacggagaggttgaacaatagcac

SEQ ID NO: 23 - DNA, 52 bp, Homo sapiens
cttcaatgatagaaacagttcttaccwccatttcagggcatatagtcagctt

SEQ ID NO: 24 - DNA, 52 bp, Homo sapien
gtttacagtgcaaaacccaggagaatmtattgaaaacaatcccaaacaataa

SEQ ID NO: 25 - DNA, 52 bp, Homo sapiens
gaccatctagacgatagaatcaacatsatcagaagaatagtgacaagaaatg

SEQ ID NO: 26 - DNA, 52 bp, Homo sapiens
tcaatccatggcaaaataaccaatgtragcaacccaagattgatatcagtct

SEQ ID NO: 27 - DNA, 52 bp, Homo sapiens
tcctccatgtgagaatacaaagagaasttagctgtttgcagcctaggagagg

SEQ ID NO: 28 - DNA, 52 bp, Homo sapiens
aaggaacttctgcttagacaattattrtgacttacttaaaatgatgaccttg

SEQ ID NO: 29 - DNA, 52 bp, Homo sapiens
actggttgctgattttgggaatccacmgtgatctctaagatacctgcaaccc

SEQ ID NO: 30 - DNA, 52 bp, Homo sapiens
ttgttatggcagttctggcaaactaayagaatccttaaacacactaagcaca

SEQ ID NO: 31 - DNA, 52 bp, Homo sapiens
agatctcaataggagtctcaactctcktagataaaaacacacaggatgatag

SEQ ID NO: 32 - DNA, 52 bp, Homo sapiens
tttagtgggagaaggagtagcttacartaatgctgtggggtggggacgtgag

SEQ ID NO: 33 - DNA, 52 bp, Homo sapiens
accacaaagtcaaatagaaattatggygtatttatgaaatagttactgaatg

SEQ ID NO: 34 - DNA, 52 bp, Homo sapiens
cattgtcttatatagaaagaaatccasaaaactattttaccttttatctctt

SEQ ID NO: 35 - DNA, 52 bp, Homo sapiens
gttaacctgttctaccaaagcttgttyttaactatcacatagtataaactcc

SEQ ID NO: 36 - DNA, 52 bp, Homo sapiens
ataaatatgggaacataccattactcyactaaattaacttatgaggtcaaaa

SEQ ID NO: 37 - DNA, 52 bp, Homo sapiens
ctatatttttctgataattttcccaaygtatttcacattcattatcataata

SEQ ID NO: 38 - DNA, 52 bp, Homo sapiens
gaaggaggggtattgtaaagtggaagwgtacttagcctgttaagtagagaaa

SEQ ID NO: 39 - DNA, 52 bp, Homo sapiens
gaatttcataggagttttattactgtragactctttttgttattatttatag

SEQ ID NO: 40 - DNA, 52 bp, Homo sapiens
ggaaaaccacttttatttatagttcaragtcatgattatgattatcagttat

SEQ ID NO: 41 - DNA, 52 bp, Homo sapiens
gtacaacttatttttttttaactggckagtaccagaagatttacaaatgtaa

SEQ ID NO: 42 - DNA, 52 bp, Homo sapiens
aaaataaaaaatgctacttctttcctrcttgagcgcacactctcatatttca

SEQ ID NO: 43 - DNA, 52 bp, Homo sapiens
ggcaagtgcatagtatattcagaggaygcaaaatggtgaatcctccatggct

SEQ ID NO: 44 - DNA, 52 bp, Homo sapiens
ccattaacaacacaggtttaaactacrcgttttcacttctatgcaaattttg

SEQ ID NO: 45 - DNA, 52 bp, Homo sapiens
ctcttttttcatgattttataattttygtatttgcaatccttaataattaac

SEQ ID NO: 46 - DNA, 52 bp, Homo sapiens
tggaatctgtatatatcagtcttagaygaatgcttagattcagatgtgggag

SEQ ID NO: 47 - DNA, 52 bp, Homo sapiens
agcattggaaacatgtatttaaggatrcactgctaactccttagctacaaga

SEQ ID NO: 48 - DNA, 52 bp, Homo sapiens
ggaaaagactcagctgtctaggatacrggattcatttactgttagccataac

SEQ ID NO: 49 - DNA, 52 bp, Homo sapiens
ccattctacttaaccactccaagtagyatttcctcaagcagaatatgctttt

SEQ ID NO: 50 - DNA, 52 bp, Homo sapiens
ttccctgatccccacagctgaaagtcwtctcactctaaacacccaactcact

SEQ ID NO: 51 - DNA, 52 bp, Homo sapiens
atgagaaagaaattcaccaggacaaayagtgttaactttaaaaattttttaa

SEQ ID NO: 52 - DNA, 52 bp, Homo sapiens
caaatcaagtgatacttattgagtagyctaccaagtgctgtggccatggaga

SEQ ID NO: 53 - DNA, 52 bp, Homo sapiens
gaccaatacacttgttccataaaaaaytcctctatattattcctagtgaaaa

SEQ ID NO: 54 - DNA, 52 bp, Homo sapiens
gaataaaacttcacgttcattttattwatttcccactccttatacatagtgc

SEQ ID NO: 55 - DNA, 52 bp, Homo sapiens
gtgagctcctttagggcaagaatcacktccaggtttcgtctcctcagtgctg

SEQ ID NO: 56 - DNA, 52 bp, Homo sapiens
atatggcacataaggttattctccttkatactgcctattgaaaaattttgag

SEQ ID NO: 57 - DNA, 52 bp, Homo sapien
tctattttccccacctcctgttggttstcctcttaaacttctccttgtgtgt

SEQ ID NO: 58 - DNA, 52 bp, Homo sapiens
caatggcttcctgcctttgtatctctmcctgcaaaatcagaagcagcgatag

SEQ ID NO: 59 - DNA, 52 bp, Homo sapien
atttaaaggtcattgtataacctttgkgcgattataataactgttactgtcc